(19) United States
(12) Patent Application Publication
(10) Pub. No.: US 2009/0287257 A1
(43) Pub. Date: Nov. 19, 2009

(54) CERVICAL PLATE

(76) Inventor: Leslie HAGEN, Cedar Park, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 12/122,104

(22) Filed: May 16, 2008

(51) Int. Cl. A61B 17/70

(52) U.S. Cl. 606/289; 606/280

(57) ABSTRACT

A cervical plate having a plurality of boreholes and one or more openings for aligning or positioning the boreholes on a vertebra. Vertices, axes and sides of the openings may be used to align the cervical plate with the midline of vertebrae and may be used to position the boreholes to ensure bone fasteners advanced through the boreholes engage cortical regions of the vertebrae. Rings positioned in the boreholes may inhibit withdrawal of the bone fasteners from the cervical plate or vertebrae after installation.

### TECHNICAL FIELD OF THE DISCLOSURE

The present disclosure generally relates to spinal fixation systems and the like. The present disclosure also generally relates to a cervical plate that includes a mechanism for fixably attaching heads of bone fasteners and openings for viewing the cortical region of the vertebra.

### BACKGROUND OF THE DISCLOSURE

Cervical plates are useful for correction of spinal deformities and for fusion of vertebrae. Typically, a cervical plate is positioned to span discs or connect vertebrae that need to be immobilized with respect to one another. Bone screws may be used to fasten a cervical plate to the vertebrae. Cervical plates are commonly used to correct problems in the cervical portion of the spine, and are often installed posterior or anterior to the spine.

Spinal plate fixation to the cervical portion of the spine may be risky because complications during surgery may cause injury to vital organs, such as the brain stem or the spinal cord. When attaching a cervical plate to a bone, bone screws are placed either bi-cortically (i.e., entirely through the vertebrae such that a portion of the fastener extends into the spinal cord region) or uni-cortically (i.e., the fastener extends into but not through the vertebrae). Uni-cortical positioning of bone screws has grown in popularity because it is generally safer to use. Bi-cortical fasteners are intended to breach the distal cortex for maximum anchorage into the bone; however, this placement of the fasteners may place distal soft tissue structures at risk. Fastener placement is particularly important in anterior cervical plate procedures because of the presence of the spinal cord opposite the distal cortex. Unfortunately, uni-cortical fasteners may move from their desired positions, because of the soft texture of the bone marrow. Further, the portion of the bone surrounding such fasteners may fail to maintain the fasteners in their proper positions. As a result, the bone fastener may progressively withdraw from the bone, also referred to as “backout.”

Backout of the fastener is particularly problematic when two fasteners are implanted perpendicular to the plate. When the fasteners are placed in such a manner, backout may occur as a result of bone failure over a region that is the size of the outer diameter of the fastener threads. Bone failure may be due to disease, injury, degeneration, or other issues. To overcome this problem, bone fasteners may be angled in converging or diverging directions with respect to each other within the bone. The amount of bone that is required to fail before backout may occur is increased by this configuration as compared to bone fasteners that are implanted in parallel. Although positioning convergent or divergent bone fasteners in a bone reduces the risk of backout, backout may still occur.

Backout may damage internal tissue structures and cause complications if the dislocated bone fastener penetrates the tissue structures. For example, if backout occurs, the bone fastener might breach the esophageal wall of the patient. Such a breach may permit bacterial contamination of surrounding tissues, including the critical nerves in and around the spinal cord. In some cases, such a breach could be fatal.

In an attempt to reduce the risk of damage to internal tissue structures, some cervical plate systems have uni-cortical fasteners that are locked to the plate. If a bone fastener withdraws from the bone, the bone fastener remains connected to the cervical plate so that it does not contact internal tissue structures. U.S. Pat. No. 5,364,399 to Lowery et al. describes one such system and is incorporated herein by reference. Lowery et al. describe a plating system that includes a locking fastener at each end of a cervical plate. The locking fastener engages the head of the bone fastener to trap the bone fastener within a recess of the plate. Since the locking bone fastener is positioned over portions of the bone screws, the locking bone fastener may extend above the upper surface of the plate. Thus, the locking bone fastener may come into contact with internal tissue structures, such as the esophagus.

Another plating system that includes a fastener-to-plate locking mechanism is the Aline™ Anterior Cervical Plating System sold by Smith & Nephew Richards Inc. in Memphis, Tenn. A description of this system can be found in the Aline™ Anterior Cervical Plating System Surgical Technique Manual by Foley, K. T. et al., available from Smith & Nephew Richards Inc., September 1996, pp. 1-16 and is incorporated herein by reference. The bone screws of this system have openings within each bone screw head for receiving a lock fastener coaxially therein. Each bone screw may be inserted into a bone such that the head of the fastener is positioned within a hole of a plate placed adjacent to the bone. The head of each bone screw is slotted so that portions of the head are deflected toward the plate during insertion of the lock fastener within the opening of the bone screw. Positioning and inserting a lock fastener within the opening can be difficult due to the small size of the lock fastener. The surgeon may be unable to hold onto the lock fastener without dropping it. If a lock fastener falls into the surgical wound, it may be difficult to retrieve. In some instances, the lock fastener may be irretrievable.

### SUMMARY OF THE DISCLOSURE

An implant system may be used to immobilize a cervical portion of a human spine. The implant system may include a cervical plate comprising openings for accommodating drills or other tools. The openings may allow a surgeon to view or access bone. The implant system may include boreholes, bone fasteners, and rings. The bone fasteners and rings may include mechanisms for anchoring or locking the bone fastener heads within the rings to inhibit backout of the bone fastener from the cervical plate.

In some embodiments of a cervical plate disclosed herein, boreholes may extend from an upper surface to a lower surface of the cervical plate. In some embodiments, the boreholes may be disposed in transversely aligned pairs at ends of the cervical plate. In some embodiments, each borehole receives at least a portion of a head of a bone fastener. Herein, “fastener” means any elongated member, threaded or non-threaded, which is securable within a bone. Bone fasteners include, but are not limited to screws, nails, rivets, trocars, pins, and barbs. In some embodiments, the bone fastener may be a bone screw. In some embodiments, a bone fastener may have a head. In some embodiments, the bone fastener head may include a portion to mate with a tool. The tool allows the insertion of the bone fastener into a bone. In some embodiments, boreholes transversely aligned at either end of the cervical plate or at one or more middle portions may also be contoured to permit the ring and/or bone fastener to be “obliquely angulated” relative to the cervical plate. Herein, “Obliquely angulated” means that the bone fastener and/or ring may be positioned throughout a wide range of angles relative to an axis normal to the cervical plate. Obliquely angulating a bone fastener into a bone may reduce the risk of backout of the bone fastener.

In some embodiments, the rings may be sized so that a ring seats within the interior space of a borehole. In some embodiments, the inner surface of each ring may be shaped to mate with the head of a bone fastener while the outer surface may be shaped based on the interior space of an end hole. In some embodiments, the outer surface of each fastener head may be tapered so that an, upper portion of the head is larger than a lower portion of the head. In some embodiments, the inner surface of the ring may have a taper that generally corresponds to the taper of the head of the bone fastener.

In some embodiments, openings may be formed through the cervical plate at various locations along a midline axis extending across the cervical plate. In some embodiments, the surface of the cervical plate that surrounds each midline hole may be tapered. Further, in some embodiments, rings positioned within the interior space of the cervical plate may have a contoured outer surface that generally corresponds to the interior space of the cervical plate. Thus, when a ring is inserted into a borehole, the shape of the interior space of the cervical plate causes the ring to remain positioned in the cervical plate in a position that is substantially normal to the plate. In some embodiments, openings may be used to visualize the implant site, intervertebral disks, cortical regions of the bone or surfaces of cervical vertebrae. In some embodiments, oblique angulation of bone fasteners positioned within the boreholes may not be required.

In some embodiments, the cervical plate may have one or more spikes located on the surface of the cervical plate that faces the spinal column. In some embodiments, spikes may be disposed at opposite ends of the cervical plate proximate the midline or boreholes. In some embodiments, the spikes may be tapped into the bone to help inhibit the cervical plate from slipping during surgical implantation.

In some embodiments, prior to surgical implantation of the spinal plate system, the rings may be placed within the bore holes of the cervical plate. The cervical plate may then be positioned adjacent to a portion of the spine that requires spinal fixation. In some embodiments, holes may be drilled and/or tapped at desired angles into portions of the bone underlying the boreholes of a cervical plate. Bone fasteners may be inserted through the boreholes into the holes in the bone. The heads of the bone fasteners may be positioned within the boreholes such that the rings surround at least a portion of the heads. In some embodiments, the rings may lock the bone fasteners in place without occupying regions outside of the boreholes.

Embodiments disclosed herein may be directed to a cervical plate having a plurality of boreholes through which the cervical plate is attachable to a vertebra via a plurality of bone fasteners and at least two openings through which a cortical region of the vertebra is visible when attaching the cervical plate to the vertebra. In some embodiments, a first and a second of the plurality of boreholes are transversely aligned at a first end of the cervical plate and a first of the at least two openings has a first tip that reaches between the first and the second of the plurality of boreholes at a first point on a midline of the cervical plate towards the first end of the cervical plate. In some embodiments, a third and a fourth of the plurality of boreholes are transversely aligned at a second end of the cervical plate and a second of the at least two openings has a second tip that reaches between the third and the fourth of the plurality of boreholes at a second point on the midline of the cervical plate towards the second end of the cervical plate.

In some embodiments, each of the first, the second, the third, and the fourth of the plurality of boreholes has an interior space that accommodates a ring with a deflectable portion through which one of the plurality of bone fasteners is insertable. In some embodiments, the ring is rotatable relative to the cervical plate within the interior space of the borehole. In some embodiments, the interior space of the borehole allows the ring to be rotated to advance a bone fastener at a selected angle relative to the cervical plate and the bone fastener may be advanced through the ring to deflect the deflectable portion of the ring outward and the bone fastener may be further advanced through the opening to allow the deflectable portion of the ring to deflect inward to inhibit withdrawal of the bone fastener from the cervical plate. In some embodiments, the deflectable portion comprises a plurality of tabs.

In some embodiments, at least one of the at least two openings has a pentagonal shape. The opening may have a base side for alignment with an inferior or superior surface of a vertebra such that alignment of the base side with an inferior or superior surface of a vertebra positions the transversely aligned boreholes within the cortical region of the vertebra. The opening may have a plurality of vertices, wherein alignment of the tip vertex relative to the midline of the vertebra aligns the cervical plate with the midline of the vertebra.

In some embodiments, at least one of the at least two openings has a rhombus shape having a major axis extending between a first vertex and a second vertex, wherein alignment of the major axis with the midline of the vertebra aligns the cervical plate with the midline of the vertebra and a minor axis extending between a third vertex and a fourth vertex such that alignment of the minor axis with an inferior or superior surface of a vertebra positions the transversely aligned boreholes within the cortical region of the vertebra.

In some embodiments, at least one of the at least two openings has a triangular shape having a base side, wherein alignment of the base side with an inferior or superior surface of a vertebra positions the transversely aligned boreholes within the cortical region of the vertebra and a tip vertex, wherein alignment of the tip vertex relative to the midline aligns the cervical plate with the midline of the vertebra.

In some embodiments, each of the first, the second, the third, and the fourth of the plurality of boreholes has an interior space that accommodates a ring with a deflectable portion through which one of the plurality of bone fasteners is insertable. In some embodiments, the ring is rotatable relative to the cervical plate within the interior space of the borehole. In some embodiments, the interior space of the borehole allows the ring to be rotated to advance the bone fastener at a selected angle relative to the cervical plate, and wherein the bone fastener may be advanced through the ring to deflect the deflectable portion of the ring outward and the bone fastener may be further advanced through the opening to allow the deflectable portion of the ring to deflect inward to inhibit withdrawal of the bone fastener from the cervical plate. In some embodiments, the deflectable portion comprises a plurality of tabs.

In some embodiments, at least one of the at least one openings has a pentagonal shape having a base side for alignment with an inferior or superior surface of a vertebra such that alignment of the base side with an inferior or superior surface of a vertebra positions the transversely aligned boreholes within the cortical region of the vertebra and a plurality of vertices, wherein alignment of a vertex relative to the midline of the vertebra aligns the cervical plate with the midline of the vertebra.

In some embodiments, at least one of the at least one openings has a rhombus shape having a major axis extending between a first vertex and a second vertex, wherein alignment of the major axis with the midline of the vertebra aligns the cervical plate with the midline of the vertebra; and a minor axis extending between a third vertex and a fourth vertex, wherein alignment of the minor axis with an inferior or superior surface of a vertebra positions the transversely aligned boreholes within the cortical region of the vertebra.

In some embodiments, an opening has a triangular shape having a base side such that alignment of the base side with an inferior or superior surface of a vertebra positions the transversely aligned boreholes within the cortical region of the vertebra and a tip vertex such that alignment of the tip vertex relative to the midline aligns the cervical plate with the midline of the vertebra.

Embodiments disclosed herein may be directed to a method for stabilizing a cervical portion of a spine, including making an incision in a patient, advancing a cervical plate into the patient via the incision, positioning the first end opening relative to a feature of a first vertebra, advancing a plurality of bone fasteners via the transversely aligned boreholes into the cortical section of the first vertebra, positioning the second end opening relative to a feature of a second vertebra and advancing a plurality of bone fasteners via the transversely aligned boreholes into the cortical section of the second vertebra. The cervical plate may include a plurality of boreholes through which the cervical plate is attachable to a vertebra via a plurality of bone fasteners and at least one opening through which a cortical region of the vertebra is visible when attaching the cervical plate to the vertebra. In some embodiments, a first and a second of the plurality of boreholes are transversely aligned at a first end of the cervical plate and at least one opening has a first vertex that reaches between the first and the second of the plurality of boreholes at a first point on a midline of the cervical plate towards the first end of the cervical plate. Each of the first and the second of the plurality of boreholes has an interior space that accommodates a ring with a deflectable portion through which one of the plurality of bone fasteners is insertable.

In some embodiments, the method comprises minimally-invasive surgery. In some embodiments, advancing a bone fastener via a transversely aligned borehole comprises advancing the ring in the borehole for advancement of the bone fastener at a selected angle relative to the cervical plate. In some embodiments, at least one of the first end opening or second end opening has a pentagonal shape. In some embodiments, the method includes aligning a first feature of the vertebra with a first side of the first end opening or a second end opening to position the transversely aligned boreholes within the cortical region of the vertebra and aligning a second feature of the vertebra with the tip vertex of the first end opening or the second end opening to align or substantially align the midline of the cervical plate with the midline of the vertebra. In some embodiments, at least one of the first end opening or the second end opening has a triangular shape. In some embodiments, the method includes aligning a first feature of the vertebra with a first side of the first end opening or the second end opening to position the transversely aligned boreholes within the cortical region of the vertebra and aligning a second feature of the vertebra with the tip vertex of the first end opening or the second end opening to align or substantially align the midline of the cervical plate with the midline of the vertebra. In some embodiments, at least one of the first end opening or second end opening has a rhombus shape and the method includes aligning a first feature of the vertebra with the major axis of the rhombus to align or substantially align the midline of the cervical plate with the midline of the vertebra and aligning a second feature of the vertebra with a second tip of the first end opening or the second end opening to position the transversely aligned boreholes within the cortical region of the vertebra.

### BRIEF DESCRIPTION OF THE FIGURES

Further advantages of the present disclosure will become apparent to those skilled in the art with the benefit of the following detailed description of embodiments and upon reference to the accompanying drawings in which:

FIG. 1 is a perspective view of one embodiment of a spine stabilization system;

FIG. 2 is a top view of one embodiment of a spinal plating system for fixation of the human spine;

FIG. 3 is a perspective view of one embodiment of a bone fastener;

FIG. 4A depicts a perspective view of one embodiment of a ring and FIGS. 4B and 4C depict cross-sectional views of embodiments of a ring;

FIGS. 5A and 5B depict partial cross-sectional views of one embodiment of a spine stabilization system;

FIG. 6 depicts perspective views of various sizes of one embodiment of a cervical plate;

FIG. 7 depicts perspective views of various sizes of one embodiment of a cervical plate;

FIG. 8 depicts perspective views of various sizes of one embodiment of a cervical plate;

FIG. 9 depicts perspective views of various sizes of one embodiment of a cervical plate;

FIG. 10 depicts perspective views of various sizes of one embodiment of a cervical plate;

FIG. 11 depicts an anterior view of one embodiment of a cervical plate attached to three vertebrae; and

FIGS. 12A-12C depict partial cross-sectional views of one embodiment of a cervical plate, illustrating one method for attaching a cervical plate to a vertebra.

While the disclosure is susceptible to various modifications and alternative forms, specific embodiments thereof are shown by way of example in the drawings and will herein be described in detail. It should be understood, however, that the drawings and detailed description thereto are not intended to limit the disclosure to the particular form disclosed, but to the contrary, the intention is to cover all modifications, equivalents and alternatives falling within the spirit and scope of the present disclosure as defined by the appended claims.

### DETAILED DESCRIPTION OF THE DISCLOSURE

The disclosure and the various features and advantageous details thereof are explained more fully with reference to the non-limiting embodiments that are illustrated in the accompanying drawings and detailed in the following description. Descriptions of well known starting materials, processing techniques, components and equipment are omitted so as not to unnecessarily obscure the disclosure in detail. Skilled artisans should understand, however, that the detailed description and the specific examples, while disclosing preferred embodiments, are given by way of illustration only and not by way of limitation. Various substitutions, modifications, additions or rearrangements within the scope of the underlying inventive concept(s) will become apparent to those skilled in the art after reading this disclosure.

As used herein, the terms “comprises,” “comprising,” includes, “including,” “has,” “having” or any other variation thereof, are intended to cover a non-exclusive inclusion. For example, a process, product, article, or apparatus that comprises a list of elements is not necessarily limited to only those elements but may include other elements not expressly listed or inherent to such process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead these examples or illustrations are to be regarded as being described with respect to one particular embodiment and as illustrative only. Those of ordinary skill in the art will appreciate that any term or terms with which these examples or illustrations are utilized encompass other embodiments as well as implementations and adaptations thereof which may or may not be given therewith or elsewhere in the specification and all such embodiments are intended to be included within the scope of that term or terms. Language designating such non-limiting examples and illustrations includes, but is not limited to: “for example,” “for instance,” “e.g.,” “in one embodiment,” and the like.

Components of spinal stabilization systems may be made of materials including, but not limited to, titanium, titanium alloys, stainless steel, ceramics, and/or polymers. Some components of a spinal stabilization system may be autoclaved and/or chemically sterilized. Components that may not be autoclaved and/or chemically sterilized may be made of sterile materials. Components made of sterile materials may be placed in working relation to other sterile components during assembly of a spinal stabilization system.

Reference is now made in detail to the exemplary embodiments, examples of which are illustrated in the accompanying drawings. Wherever possible, the same reference numbers will be used throughout the drawings to refer to the same or like parts (elements).

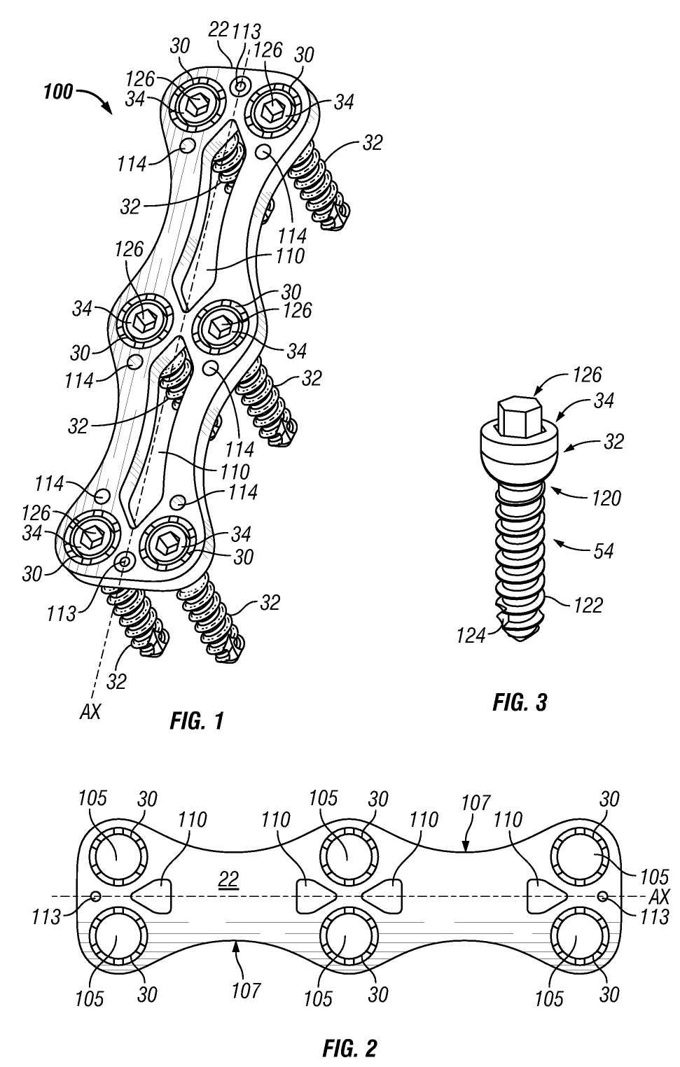

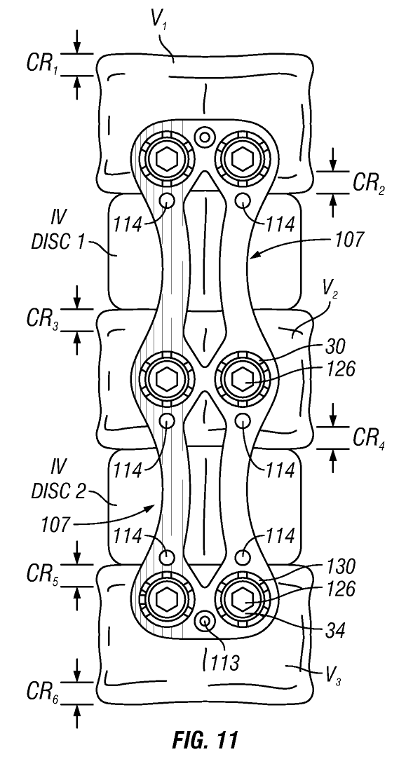

FIG. 1 depicts a perspective view of one embodiment of spine stabilization system 100 including cervical plate 22 having bone fasteners 32 with heads 34 advanced and seated within rings 30. In some embodiments, cervical plate 22 has symmetrical features along a longitudinal axis AX. In some embodiments, openings 110 may be sized and shaped to enable a surgeon to align cervical plate 22 with the midline of one or more vertebrae, to position cervical plate 22 such that bone fasteners 32 are positioned within the cortical region of the vertebral body, and intervertebral discs are visible (see FIG. 11). In some embodiments, cervical plate 22 may include guide openings 114, which may be used as a guide for a drill or other tool. In some embodiments, each of openings 114 is positioned at about an equal distance from opening 110 and borehole 105. Cervical plate 22 may be used to correct problems in the cervical portion of the spine. Cervical plate 22 may be installed anterior to the spine. Cervical plate 22 shown in FIG. 1 may be placed adjacent to a portion of the spine and connect to three vertebrae (i.e., span least two vertebral levels).

FIG. 2 depicts cervical plate 22 having boreholes 105 transversely aligned at either end. Cervical plate having sets of transversely aligned boreholes 105 located at either end of cervical plate may be useful for a single-level spine stabilization. Cervical plate 22 may also include boreholes 105 transversely aligned at some middle portion, which may be useful for two-level spine stabilization. Cervical plate 22 may further include openings 110 located on the midline of cervical plate 22. Boreholes 105 may include rings 30. Cervical plate 22 may include two transversely aligned boreholes 105 through which bone fasteners may be inserted to attach cervical plate 22 to a first vertebra (not shown). Cervical plate 22 may include two transversely aligned boreholes 105 through which bone fasteners 32 may be inserted to attach cervical plate 22 to a second vertebra (not shown). Cervical plate 22 may include two transversely aligned boreholes 105 through which bone fasteners 32 may be inserted to attach cervical plate 22 to a third vertebra (not shown). Rings 30 in boreholes 105 may retain bone fasteners 32 in cervical plate 22. Cervical plate 22 may include one or more openings 110 for improved visualization of the implant site. Openings 110 allow a surgeon to view anatomical landmarks or other indicators for aligning or substantially aligning cervical plate 22 on vertebrae or for positioning bone fasteners in vertebrae. Cervical plate 22 may be shaped for improved visibility or handling. In some embodiments, cervical plate 22 may include openings 113. In some embodiments, openings 113 may be located on the longitudinal axis of cervical plate 22. In some embodiments, neck regions 107 may provide increased visibility of the implant site, provide tactile sensation to facilitate handling by the surgeon, or other benefits.

FIG. 3 depicts one embodiment of bone fastener 32. Each of bone fasteners 32 may be inserted through boreholes 105 in cervical plate 22. Bone fastener 32 may include shank 54, head 34, and neck 120. Shank 54 may include threading 122. In some embodiments, threading 122 may include self-tapping start 124. Self-tapping start 124 may facilitate insertion of bone fastener 32 into vertebral bone. Head 34 of bone fastener 32 may include various tool portions 126 to engage a driver that inserts bone fastener 32 into a vertebra. In some embodiments, the driver may also be used to remove an installed bone fastener 32 from a vertebra. Tool portions 126 may include recesses and/or protrusions designed to engage a portion of the driver. In some embodiments, bone fastener 32 may be used in a minimally invasive surgery (“MIS”). In some embodiments, cannulated bone fasteners 32 may accommodate a tool for insertion during a minimally invasive surgery.

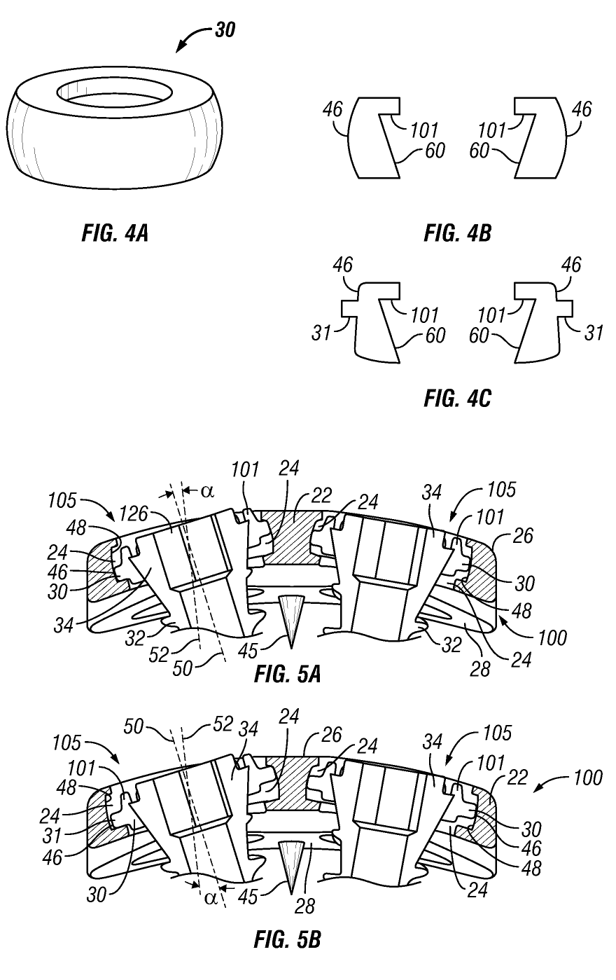

FIG. 4A depicts a perspective view of one embodiment of ring 30 and FIGS. 4B and 4C depict cross-sectional views of embodiments of ring 30. Rings 30 may be positioned in boreholes 105 of cervical plate 22. Angulation of rings 30 in boreholes 105 in cervical plate 22 may allow fine adjustment of the engagement angle of bone fasteners 32. In addition, angulation of rings 30 in boreholes 105 may allow adjustment in the orientation of bone fasteners 32 to attach cervical plate 22 to the cortical region of the bone while still allowing cervical plate 22 to be positioned on the midline of the spine. Ring 30 may include deflectable portions 101. Deflectable portions 101 may include tabs or teeth that may allow shank 54 of bone fastener 32 to pass through ring 30. In some embodiments, deflectable portions 101 may prevent head 34 or a portion of head 34 from passing through ring 30. In some embodiments, deflectable portions 101 may inhibit head 34 or a portion of head 34 from withdrawing from ring 30 and cervical plate 22.

As depicted in FIG. 4B, ring 30 may have arcuate outer surface 46. Inner surface 60 may contact head 34 of bone fastener 32. Outside surfaces 46 of rings 30 may have arcuate or spherical contours that substantially correspond to the contours of the inner surfaces of boreholes 105. Having a contoured ring outer surface 46 that substantially corresponds to an inner surface of boreholes 105 allows bone fasteners 32 to be capable of polyaxial rotation within boreholes 105. As depicted in FIG. 4C, outer surface 46 of ring 30 may include flange 31 that substantially corresponds to an inner surface of borehole 105. Inner surface 60 may contact head 34 of bone fastener 32.

FIGS. 5A-5B depict cross-sectional views of embodiments of spine stabilization systems 100 for stabilizing a portion of a cervical spine, in which bone fasteners 32 for attaching cervical plate 22 to a vertebra may be inserted in rings 30 contained in interior space 24. In some embodiments, ring 30 may be inserted and positioned into an interior space 24 in borehole 105 in plate 22.

In some embodiments, the ability of ring 30 to rotate polyaxially within interior space 24 of borehole 105 allows bone fasteners 32 to be positioned through cervical plate 22 at various angles with respect to an axis that is normal to cervical plate 22. FIGS. 5A and 5B show angle α (Alpha) for particular fastener configurations. Angle α (Alpha) is defined between the longitudinal axis 50 of bone fastener 32 and axis 52 aligned normal to cervical plate 22. In some embodiments, the angle α (Alpha) may range from 0 to about 45 degrees. In some embodiments, the angle α (Alpha) may range from about 0 to about 30 degrees. In some embodiments, the angle α (Alpha) may range from 0 to about 0 and 15 degrees. Bone fasteners 32 may also be set in boreholes 105 such that bone fasteners 32 are non-planar with respect to a latitudinal plane extending through boreholes 105 in plate 22. For example, a first bone fastener 32 may be positioned in a first borehole 105 and angled in a first orientation (e.g., out of the page) and a second bone fastener 32 may be positioned in a second borehole 105 and angled in a second orientation (e.g., into the page). Bone fasteners 32 set in diverging or converging directions in the interior spaces 24 may reduce the possibility of backout. Further, the use of rings 30 to fixedly attach bone fasteners 32 to plate 22 may inhibit damage to tissue structures by any bone fasteners 32 that do loosen within a bone, since such bone fasteners 32 would remain attached to cervical plate 22. Bone fasteners 32 may be placed in uni-cortical positions within a bone since the problem of fastener backout is reduced by having obliquely angulated fasteners in converging or diverging configurations.

Interior spaces 24 of boreholes 105 may be contoured to define the limits of angulation of head 34 with respect to cervical plate 22. Inner surfaces 48 of boreholes 105 may have arcuate or spherical contours. Head 34 of bone fastener 32 may have a tapered profile. Head 34 of bone fastener 32 may have various taper configurations and/or texturing to enhance coupling of bone fastener 32 with ring 30. As depicted in FIG. 5A, in some embodiments, head 34 may be contoured such that deflectable portions 101 of ring 30 contact bone fastener 32 to prevent bone fastener 32 from backing out of cervical plate 22. Also, borehole 105 may be sized to prevent bone fastener 32 from passing completely through cervical plate 22. In some embodiments, neck 120 of bone fastener 32 may have a smaller diameter than adjacent portions of head 34 and shank 54. The diameter of neck 120 may fix the maximum angle that bone fastener 32 can be rotated relative to cervical plate 22. In some embodiments, the diameter of neck 120 may be small enough to ensure that ring 30 positioned in cervical plate 22 determines the maximum angle of attachment of cervical plate 22 to its area. Angulation of rings 30 in boreholes 105 in cervical plate 22 may allow fine adjustment of engagement angles of bone fasteners 32. In addition, angulation of rings 30 in boreholes 105 may allow adjustment in the orientation of bone fasteners 32 to attach cervical plate 22 to the cortical region of the bone while still allowing cervical plate 22 to be positioned on the midline of the spine.

Ring 30 may have inner surface 60 for mating with head 34, as depicted in FIGS. 5A and 5B. In one embodiment, the bottom portion of head 34 may be smaller than the upper portion of an unstressed ring 30, while the upper portion of head 34 may be larger than the upper portion of ring 30. As fastener 32 passes through ring 30, head 34 applies a radial force to deflectable portions 101 of ring 30 which causes deflectable portions 101 to expand within interior space 24. In some embodiments, expanding ring 30 may cause outer surface 46 of ring 30 to abut against inner surface 48 of interior space 24 such that an interference fit forms between fastener head 34, ring 30, and plate 22. Thus, ring 30, bone fastener 32 and cervical plate 22 may fit together such that each element obstructs the movement of the other elements. Hoop stress of ring 30 on head 34 may fixedly attach bone fastener 32 to cervical plate 22.

In some embodiments, ring 30 may be capable of rotating within interior space 24 of cervical plate 22 such that one portion of ring 30 is adjacent to the upper surface 26 of cervical plate 22 while another portion of ring 30 is adjacent to the lower surface 28 of cervical plate 22. The width and thickness of deflectable portions 101 may determine the range of motion of ring 30 within cervical plate 22. In some embodiments, deflectable portions 101 may have an increased thickness or interior space 24 may be smaller to limit the range of motion of ring 30. In some embodiments, deflectable portions 101 may have a decreased thickness or interior space 24 may be larger to limit the range of motion of ring 30. FIG. 5A depicts ring 30 having deflectable portions 101 having a selected width and thickness inside interior space 24 having a selected size. FIG. 5B depicts ring 30 having deflectable portions 101 having a selected width and thickness and interior space 24 having a selected size. By changing one or more of the width and thickness of deflectable portions 101 and the size of interior space 24, a range or motion for ring 30 may be established. In one embodiment, ring 30 may be retained within interior space 24 without extending beyond the upper surface 26 or lower surface 28 of cervical plate 22. Ring 30 and fastener head 34 remain within interior space 24 so that spine stabilization system 100 may have a minimal profile width. Having rings 30 and fastener heads 34 which do not extend above the upper surface 26 or below the lower surface 28 of cervical plate 22 may prevent rings 30 and heads 34 from contacting adjacent tissue structures. In some embodiments, bone fasteners 32 can be angulated relative to cervical plate 22 such that rings 30 extend from interior spaces 24 beyond upper surface 26 and/or lower surface 28 of cervical plate 22.

In one embodiment, ring 30 has an outer width that is less than or about equal to the width of an interior space 24 in cervical plate 22 at a location between an upper surface 26 and lower surface 28 of the bone plate. The width of each interior space 24 proximate upper surface 26 and lower surface 28 of cervical plate 22 is less than or about equal to an outer width of ring 30. The width of ring 30 may inhibit ring 30 positioned in interior space 24 from accidentally falling or being pulled out of borehole 105. Prior to surgery, ring 30 may be positioned within each interior space 24 of cervical plate 22. When seated within interior space 24, ring 30 may be capable of swiveling within borehole 105, but ring 30 is inhibited from falling out of borehole 105 because of reduced width of borehole 105 proximate upper surface 26 and lower surface 28, 28 of cervical plate 22. A surgeon may use cervical plate 22 having rings 30 positioned within borehole 105 prior to surgery. U.S. Pat. Nos. 6,030,389, 6,331,179, 6,454,769 and 6,964,664 disclose systems and methods utilizing rings 30 in a plate and are hereby incorporated by reference.

Also shown in FIGS. 5A and 5B, in some embodiments, cervical plate 22 may include spike 45 or barb for engaging a vertebra. Cervical plate 22 may have one or more spikes 45 located on surface 28 of cervical plate 22 for engagement with the bone. Spikes 45 may be disposed in pairs at opposite ends of the cervical plate 22 proximate the interior spaces 24. The spikes 45 may be tapped into the bone to help inhibit the cervical plate 22 from slipping during surgical implantation.

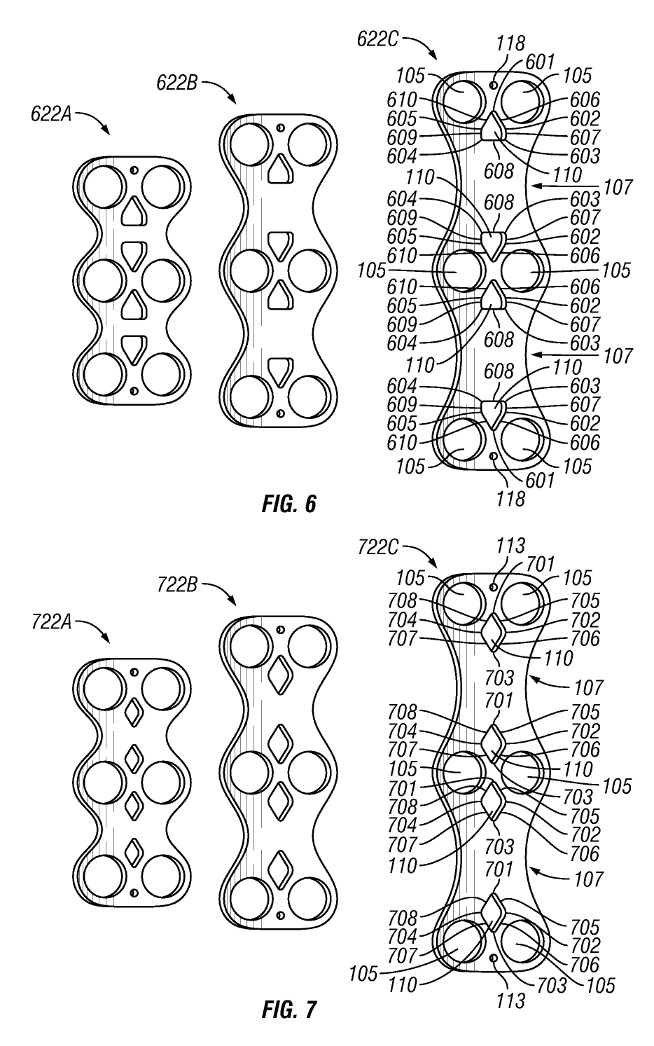

FIGS. 6-10 depict embodiments of various configurations of cervical plates 22 having openings 110 for improved visualization of the vertebra, which may be useful for aligning cervical plate 22 with a superior or inferior surface of a vertebra and/or aligning cervical plate 22 with the midline of a vertebra.

FIG. 6 depicts two-level cervical plates 622A-622C having different overall lengths and having pentagonal shaped openings 110 with five vertices 601-605 and five sides 606-610. Sides 606 and 610 may have a first length, sides 607 and 609 may have a second length, and side 608 may have a third length, or all sides 606-610 may have an identical length. By adjusting the length of sides 606-610, opening 110 may be configured to provide more visual clues for attaching cervical plate 22 to a vertebra. For example, the height of pentagonal opening 110 may be such that a surgeon may align base side 608 relative to a superior and/or inferior surface of a vertebra to position boreholes 105 within the cortical region of the vertebra, sides 607 and 609 may define a distance from the inferior or superior surface of the vertebra that the surgeon wishes to avoid inserting bone fastener 32, sides 606 and 610 may define a preferred region for implanting bone fasteners 32. Similarly, the angles of vertices 601-605 may be used to assist a surgeon attaching cervical plate 22 to a vertebra. For example, a surgeon may use tip vertex 601 to align cervical plate 22 with the midline of a vertebra, vertices 602-605 may be used to ensure cervical plate 22 is aligned with a superior or inferior surface of a vertebra, or the like.

FIG. 7 depicts two-level cervical plates 722A-722C having openings 110 having vertices 701-704 and sides 705-708 forming a major axis along a longitudinal axis of cervical plate 22 (i.e., intersecting vertices 701 and 703) and a minor axis substantially perpendicular to the major axis (i.e., intersecting vertices 702 and 704). In some embodiments, the major axis of opening 110 is the midline of cervical plate 22. In some embodiments, aligning the major axis of opening 110 with the midline of a vertebra aligns cervical plate 22 with the midline of the vertebra. In some embodiments, positioning cervical plate 22 such that the minor axis of opening 110 is aligned relative to a superior or inferior surface of a vertebra ensures that openings 105 are positioned for advancing bone fasteners 32 into the cortical region of the vertebra. In some embodiments, a tool (not shown) may be temporarily positioned in opening 110 near vertex 703 during implantation of cervical plate 22. Cervical plate 22 may be moved until the tip of the tool contacts the surface of a vertebra. Positioning of cervical plate 22 may be confirmed by looking through opening 110 near vertex 701 or vertices 702 and 704 to identify cortical regions of bone. Once cervical plate 22 has been attached to the vertebra, the tool may be removed.

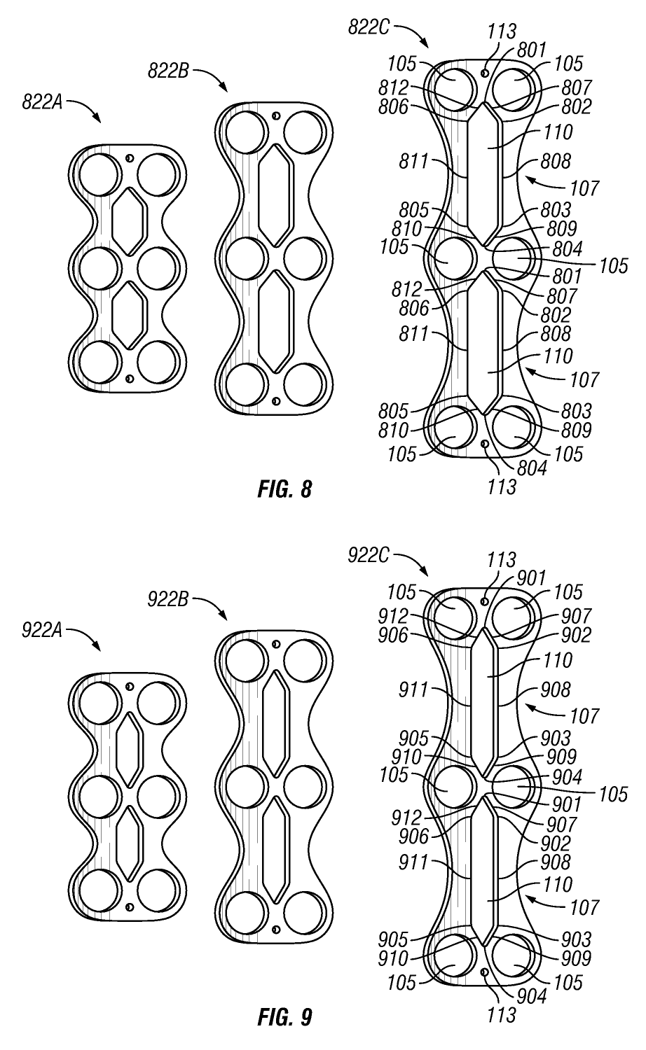

FIGS. 8-10 depict various open configurations, which may further allow a surgeon to visually inspect the vertebral disc. FIG. 8 depicts perspective views of cervical plates 822A-822C in which visualization of a superior surface of a first vertebra and the inferior surface of a second vertebra may be possible through a single opening 110. Plates 822A-822C include openings 110 formed from vertices 801-806 and sides 807-812. A major axis may be defined to pass through vertices 801 and 804, and two minor axes may be defined to pass through vertices 802 and 806 and through vertices 803 and 805. Vertices 801 and 804 may be used to align cervical plates 822A-822C with the midline of one or more vertebrae. Vertices 802 and 804 may be used to position cervical plate 822B relative to a superior or inferior surface of a first vertebra to ensure boreholes 105 are positioned such that bone fasteners 32 may be advanced through boreholes 105 into the cortical region of the first vertebra. Vertices 803 and 805 may be used to position cervical plate 822B relative to an inferior or a superior surface of a second vertebra to ensure boreholes 105 are positioned such that bone fasteners 32 may be advanced through boreholes 105 into the cortical region of the second vertebra. Opening 110 may still allow a surgeon to visually inspect an intervertebral disc. Plate 822C may have opening 110 shaped similarly to opening 110 on cervical plates 822A or 822B but having longer sides 808 and 811. An advantage to having longer sides 808 and 811 may be the ability to accurately align cervical plate 22 with the midline of a vertebra.

FIG. 9 depicts perspective views of cervical plates 922A-922C in which visualization of a superior surface of a first vertebra and the inferior surface of a second vertebra may be possible through a single opening 110. Plates 922A-922C may be similar to plates 822A-822C in FIG. 8 but opening 110 may be narrower. A narrower opening 110 may result in cervical plate 22 having more rigidity, torsional stiffness, tensile strength, or some other desired mechanical property. A wider opening 110 (such as depicted in FIG. 8) may provide greater access to the intervertebral disc, superior and/or inferior surfaces of the vertebrae, or greater flexibility of cervical plate 22. Sides 932 and 933 may be tapered (shown) such that cervical plates 922A-922C may have a “neck” portion, or may be straight.

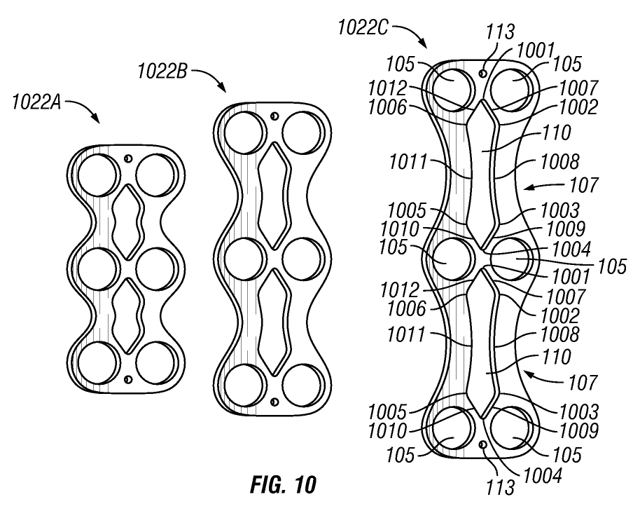

FIG. 10 depicts perspective views of cervical plates 1022A-1022C in which visualization of a superior surface of a first vertebra and the inferior surface of a second vertebra may be possible through a single opening 110 formed by sides 1007-1012 joined at vertices 1001-1006. As shown in FIG. 10, opening 110 in cervical plates 1022A-1022C may have tapered sides 1008 and 1011. Tapered opening 110 may provide a surgeon with greater visibility near boreholes 105 but with increased stiffness, torsional stability, or the like due to cervical plate 22 having greater thickness or the geometry of opening 110. Cervical plate 1022C may be similar to cervical plates 1022A and 1022B but have longer dimensions. Openings 110 in cervical plates 1022A, 1022B and 1022C may have proportionately longer dimensions or may be asymmetric. For example, if boreholes 105 located in a middle portion of cervical plates 1022A-1022C are aligned with the center of a vertebral body (such as depicted in FIG. 11, discussed below), bone fasteners 32 advanced through boreholes 105 may not contact the cortical region of the vertebra and it may not be necessary for sides 1009 and 1010 or 1007 and 1012 to extend between boreholes 105 located in the middle portion.

FIG. 11 depicts an anterior view of one embodiment of cervical plate 22 attached to a portion of the spine. In some embodiments, boreholes 105 may be positioned within cortical region CR₂ on cervical vertebra V₁, boreholes 105 in cervical vertebra V₂ may not be positioned within cortical regions CR₃ or CR₄, and boreholes 105 on cervical vertebra V₃ may be positioned within cortical region CR₅. Cervical plate 22 having openings 110 allows a surgeon to see intervertebral discs IV₁ and IV₂, inferior surfaces of V₁ and V₂, superior surfaces of cervical vertebrae V₂ and V₃, the midline of cervical vertebrae V₁V₂ and V₃, and at least portions of cortical regions CR₂, CR₃, CR₄ and CR₅. Cervical plate 22 may have tapered portions 107 for improved visibility of intervertebral discs IV₁ and IV₂. Cervical plate 22 may have openings 113 for inserting temporary fixation pins in lieu of spikes. Cervical plate 22 may include openings 114 for receiving a portion of a tool. In some embodiments, positioning a drill guide tip in openings 114 allows a surgeon to tap or drill a vertebra for advancement of bone fasteners 32.

In one embodiment, a spinal stabilization system is prepared for surgical implantation by positioning rings 30 within interior spaces 24. Shank 54 of each bone fastener 32 may be advanced through each ring 30. As head 34 of bone fastener 32 is advanced through ring 30 positioned in interior space 24 of cervical plate 22, bone fastener 32 may deflect deflectable portions 101 radially outward. Once head 34 of bone fastener 32 passes deflectable portions 101, deflectable portions 101 may return to an original undeflected state. An incision is made in the patient and cervical plate 22 having rings 30 positioned in boreholes 105 and bone fasteners 32 positioned in rings 30 is advanced through the incision. Cervical plate 22 is positioned over the vertebrae and bone fasteners 32 are advanced into the cortical region of the vertebrae to attach cervical plate 22 to the vertebrae.

In some embodiments, minimally invasive surgery (MIS) procedures may be used to implant cervical plate 22 or bone fasteners 32. Minimally invasive procedures may involve locating a surgical site and a position for a single skin incision to access the surgical site. The incision may be located above and between (e.g., centrally between) vertebrae to be stabilized. An opening under the skin may be enlarged to exceed the size of the skin incision. Movement and/or stretching of the incision, advancing cervical plate 22 and bone fasteners 32 independently, or other techniques may allow the length of the incision and/or the area of a tissue plane to be minimized. In some embodiments, minimally invasive insertion of a spinal stabilization system may not be visualized. In certain embodiments, insertion of a spinal stabilization system may be a top-loading, mini-opening, muscle-splitting, screw fixation technique.

Various techniques may be used to plan the skin incisions and entry points. In one embodiment, the planning sequence for a single-level stabilization may include the following four steps. First, an anteroposterior image may be obtained of the target vertebral bodies. Second, horizontal lines may be marked on the patient. Third, an oblique or “bullseye” view may be obtained on each side of the patient for each vertebra that is to be stabilized. Fourth, an incision may be made in the skin between along the vertical oblique view lines. The skin incision may be from about 2 cm to, about 4 cm long. In some embodiments, the incision may be from about 2.5 cm to about 3 cm long. Limiting the length of the incision may enhance patient satisfaction with the procedure. The incisions may be pre-anesthetized with, for example, 1% lidocaine with 1:200,000 epinephrine. To blunt the pain response, a long spinal needle may be used to dock on the bone entry point and inject the planned muscle path in a retrograde fashion as well. Once the incision has been made, tissue surrounding the incision may be pulled and/or stretched to allow access to a target location in a vertebra.

A scalpel may be used to make a stab wound. In one embodiment, the scalpel may be a #11 scalpel. In one embodiment, a tissue wedge may be advanced into the patient to the target vertebrae. The tissue wedge may be wanded toward the target location at the second vertebra, thereby creating a plane in muscle and other tissue between the first and second vertebrae. The wanding action may be repeated more than once (e.g., two or three times) to create a good working plane and displace unwanted tissue from the plane. The wanding may create a tissue plane. In some embodiments, the tissue plane may be substantially trapezoidal. In certain embodiments, a tissue plane may be created before cervical plate 22 is inserted into the patient.

Cervical plate 22 may be passed through the incision towards the vertebrae to be stabilized. In some embodiments, cervical plate 22 may be advanced through the incision to the target vertebrae. In some embodiments, cervical plate 22 may be advanced longitudinally and then rotated into position. In some embodiments, a first end of cervical plate 22 may be advanced toward a first vertebra and then translated to a second vertebra. In some embodiments, a first end of cervical plate 22 may be advanced to a first vertebra and then the second end of cervical plate 22 may be rotated to a second vertebra. Cervical plate 22 may be advanced by first advancing a tool or guide into the patient and then advancing cervical plate 22 using the tool or guide, or may be advanced manually.

In some embodiments, cervical plate 22 may be guided via openings 113 on cervical plate 22. The position of cervical plate 22 on a vertebra may be checked for positioning and alignment. In some embodiments, the position of cervical plate 22 may be changed to ensure bone fasteners 32 will engage the cortical region of the vertebra.

In some embodiments, cervical plate 22 may have a spike 45 or barb instead of openings 113. Cervical plate 22 may be advanced via boreholes 105, openings 110. Cervical plate 22 may be aligned with the midline of the vertebrae and the spike 45 or barb may be advanced into the vertebra to position cervical plate 22 adjacent to the vertebrae until bone fasteners 32 can attach cervical plate 22 to the vertebrae.

A surgeon may utilize openings 110, boreholes 105, openings 113, spikes 45, tapered regions 107 or any combination thereof to align and position cervical plate 22 on the vertebrae. In some embodiments, cervical plate 22 may have tapered regions 107 between sets of transversely aligned boreholes 105, which may provide tactile clues. Tactile clues that communicate where the surgeon is holding cervical plate 22 along with visual indicators such as seeing a surface of the vertebra through opening 110 may result in improved positioning of bone fasteners 32 in cortical regions of vertebrae, improved angulation of bone fasteners 32 in cervical plate 22 to prevent backout, improved alignment of the vertebrae, and other advantages.

In some embodiments, openings 110 may have a pentagonal shape, such as opening 110 depicted in FIG. 6. In some embodiments, opening 110 may have sides 606-610 of equal length and equal angles. In some embodiments, sides 606, 607, 608, 609 and/or 610 may differ in length. In some embodiments, the length of sides 606, 607, 608, 609 and/or 610 and angles of vertices 601-605 may be selected to provide additional cues for visualization, alignment or positioning of cervical plate 22. For example, in some settings it may be desirable to have sides 607 and 609 of shorter length such that base side 608 may be used in conjunction with vertices 602 and 605 to align cervical plate 22 with a surface of a vertebra. Those skilled in the art will appreciate that the angles of vertices 601, 602, 603, 604 and/or 605 may be any angle and the length of each of sides 606, 607, 608, 609 and/or 610 may be any length to form opening 110 to provide additional cues for the placement of opening 110 with respect to a surface of a vertebra, the midline of the vertebra, or some other desired placement. In one embodiment, the length of sides 606, 607, 608, 609 and/or 610 may be of selected length such that when vertices 603 and 604 are aligned with a surface of a vertebra, boreholes 105 are positioned relative to the cortical region of the vertebra.

In some embodiments, opening 110 may be used to ensure a desired position of cervical plate 22. In one embodiment, opening 110 may have a triangular shape. In one embodiment, opening 110 may be formed sides such as sides 607 and 609 in FIG. 6 with lengths short enough to approximate a triangular shape. Positioning of cervical plate 22 may be established using a base side of opening 110. Base side 608 may be positioned on a superior vertebra by aligning base side 608 with the inferior surface of the superior vertebra. Base side 608 may be positioned on an inferior vertebra by aligning base side 608 with the superior surface of the inferior vertebra. By aligning the base side of opening 110 with the appropriate surface, the surgeon may determine that bone fasteners 32 advanced through boreholes 105 will engage cortical bone. Alignment of cervical plate 22 having a triangular shape may be possible using tip vertex 601 of opening 110. Aligning tip vertex 601 with an anatomical landmark of the midline may ensure cervical plate 22 is aligned with the midline. In some embodiments, tip vertex 601 may be used in conjunction with sides 606 and 610 to align cervical plate 22 with the midline of the vertebra. Those skilled in the art will appreciate that the length of any side and the angle of any vertex may be based on preferences. For example, it may be easier for a surgeon to align base side 608 with a surface of a vertebra and the surgeon may have more difficulty aligning tip vertex 601 with the midline of the vertebra. In that case, opening 110 may have a shorter base side 608 and longer side sides 606 and 610, such that tip vertex 601 has an acute angle. Similarly, if the surgeon has difficulty aligning base side 608 with a surface of the vertebra, base side 608 may be longer and tip vertex 601 may form an obtuse angle.

In some embodiments, opening 110 may have a rhombus shape, such as depicted in FIG. 7, having a major axis and a minor axis. For example, the major axis may extend between vertices 701 and 703, and the minor axis may extend between vertices 702 and 704. In some embodiments the minor axis may be used to align cervical plate 22 for ensuring that bone fasteners 32 advanced through boreholes 105 are positioned within the cortical region of a vertebra. In one embodiment, positioning vertices 702 and 704 relative to an inferior surface of a superior vertebra may align the minor axis of opening 110 such that bone fasteners advanced through boreholes 105 engage a cortical region of the superior vertebra. Similarly, positioning vertices 702 and 704 relative to a superior surface of an inferior vertebra may align the minor axis of opening 110 such that bone fasteners advanced through boreholes 105 engage a cortical region of the inferior vertebra. In some embodiments, aligning the major axis of opening 110 along the midline of a vertebra aligns cervical plate 22 with the midline of the vertebra. Those skilled in the art will appreciate that the shape of opening 110 may be based on the length of the sides and the angles of the vertices. In some embodiments, cervical plate 22 may be easier to align with the midline of the vertebra if the major axis is longer. By changing the angle of vertices 701, 702, 703 and/or 704 or the length of sides 705, 706, 707 and/or 708 of opening 110, the major axis may be lengthened. In some embodiments, cervical plate 22 may be easier to align with the midline of the vertebra if the minor axis is longer and the surgeon orients the minor axis perpendicular to the midline of the vertebra. By changing the angle of vertices 701, 702, 703 and/or 704 of opening 110, the minor axis of opening 110 may be lengthened.

In some embodiments, insertion of a spinal stabilization system may include gradually increasing the diameter of an opening formed in a vertebral body to accept bone fastener 32. In some embodiments, a targeting needle may have an outer diameter of about D. In some embodiments, a bone awl inserted after the targeting needle may have an outer diameter incrementally larger than the outer diameter of the targeting needle. As used herein, an incrementally larger diameter May be large enough to allow a snug but adjustable fit. For example, the bone awl may have outer diameter of about (D+x). A tap portion of a bone tap inserted after the bone awl may have a minor diameter of about (D+2x). Bone fastener 32 may have a minor diameter of about (D+3×). In some embodiments, x may be between about 0.1 mm and about 1.0 mm. For example, x may be about 0.5 mm. Incremental sizing of the targeting needle, the bone awl, the tap, and bone fastener 32 may promote a proper fit of bone fastener 32 in the vertebra to be stabilized.

In some embodiments, a tool that has been inserted through opening 110 may be used to locate or position cervical plate 22 relative to the vertebrae. In some embodiments, once cervical plate 22 contacts the bony anatomy of a vertebra, cervical plate 22 may be “walked” along medially or distally along the midline of the vertebrae to ensure boreholes 105 are positioned over the cortical region of the vertebra or “walked” laterally to ensure that a vertex or major axis of cervical plate 22 is aligned with the midline of the vertebrae. In some embodiments, spike 45 may be advanced into the vertebral body. Spike 45 may be used for temporarily holding cervical plate 22 in position until bone fasteners 32 are advanced or may provide additional holding forces to complement bone fasteners 32 in openings 105.

In some embodiments, once cervical plate 22 has been positioned on the vertebrae, a surgeon may attach cervical plate 22 to the vertebrae. In some embodiments, rings 30 may be positioned within each interior space 24 before the surgical procedure.

Bone fasteners 32 may be positioned through rings 30. A driver having tool portions for engaging tool portions 126 on head 34 of bone fastener 32 may be used to advance bone fasteners 32 through boreholes 32 in cervical plate 22. Bone fastener 32 may then be rotated to insert bone fastener 32 into a bone. As bone fastener 32 is advanced through ring 30, fastener head 34 moves into ring 30. Movement of head 34 into ring 30 causes deflectable portions 101 to radially expand. In some embodiments, movement of head 34 into ring 30 causes ring 30 to expand against interior space 24 to fix bone fastener 32 relative to cervical plate 22. Once bone fastener 32 is advanced through borehole 105 and engaged in cortical bone of a vertebra, the driver may be removed. Fasteners 100 may be inserted through the remaining interior spaces 24 and into bone to securely attach cervical plate 22 to the bones.

After a threaded passage of a desired length has been formed in the cortical portion of the vertebral body, a second measurement of the position of the tap. A length of bone fastener 32 may be determined by taking a difference between the first and second measurements. In some embodiments, an estimate of length may be derived based upon fluoroscopic images and a known length of the tap that is visibly recognizable in the fluoroscopic images. The tap may be removed from the cortical portion of the vertebral body by rotating the tap.

Bone fasteners 32 of an appropriate length may be selected for insertion in a patient. The size of bone fastener 32 may be verified with measurement indicia in an instrumentation set. In some embodiments, measurement indicia may be etched or printed on a portion of an instrumentation set. For example, the chosen bone fastener embodiment may be placed over the outline of a bone fastener embodiment printed on a tray of the instrumentation set.

The chosen bone fastener 32 may be attached to an insertion/extraction tool. In one embodiment, tool portions 126 of bone fastener 32 may be engaged by a driver head. When bone fastener 32 is coupled to the driver, a drive portion of the driver may be coupled to a tool portion of bone fastener 32. In some embodiments, a handle may be attached to the shaft of the fastener driver after bone fastener 32, the detachable member, and the fastener driver combination is positioned down the guide wire through the dilator and oriented to the cortical portion of the vertebra.

In some embodiments, an insertion/extraction tool may be used to advance bone fastener 32 into the vertebral body. The tool may be inserted along a guide wire into openings 114 in cervical plate 22. In some embodiments, tissue surrounding the incision may be pulled and/or stretched to allow a desired angular orientation of bone fastener 32 relative to a vertebral body.

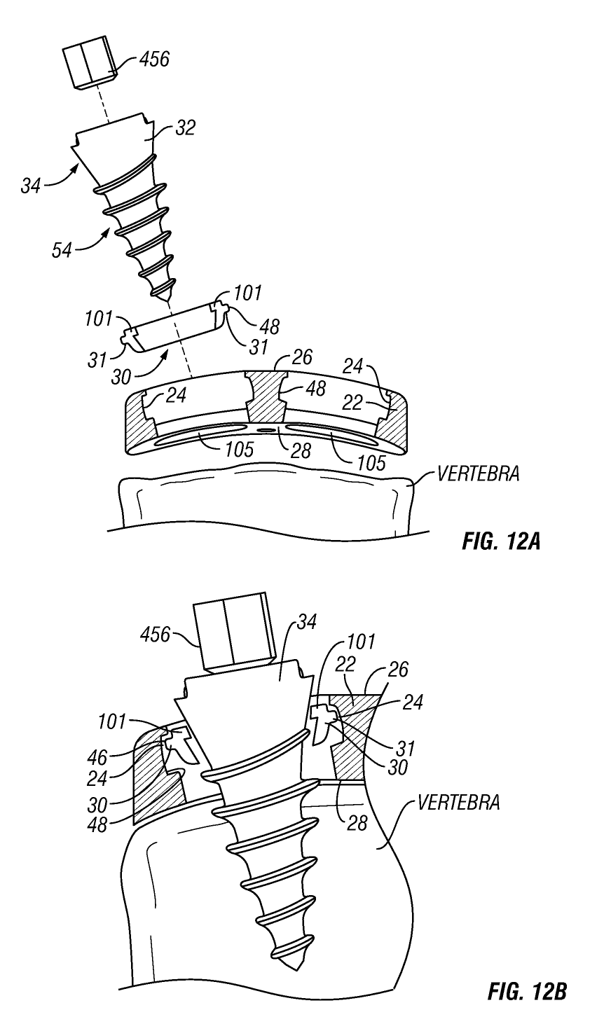

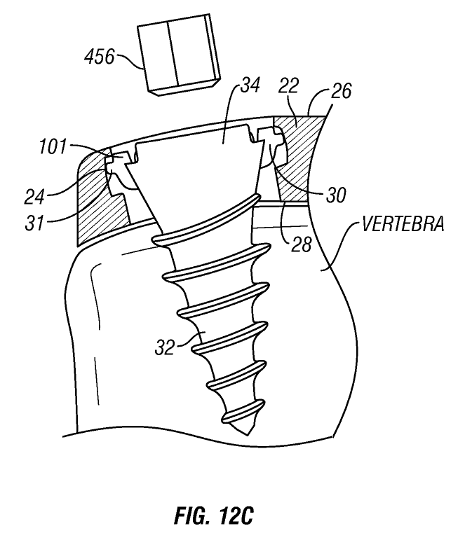

FIGS. 12A-12C show partial cross sectional views of bone fastener 32, ring 30, cervical plate 22, and driver head 456 of an insertion/extraction tool (not shown) during the insertion and extraction processes. As shown in FIGS. 12A-12C, cervical plate 22 may have a curvature. In some embodiments the curvature may enhance fixation of cervical plate 22 to a bone.

FIG. 12A depicts, an exploded cross-sectional view of components of a spine stabilization system. Cervical plate 22 may have boreholes 105 having interior space 24 contoured to accommodate ring 30. Boreholes 105 may have a diameter through which shank 54 of bone fastener 32 may be advanced. Head 34 of bone fastener 32 may accommodate driver head 456 of a tool. Ring 30 may have flanges 31 for engaging interior space 24.

Referring to FIG. 12B, driver head 456 of an insertion/extraction tool may be inserted in tool portion 126 of head 34. Ring 30 may be positioned inside interior space 24 in cervical plate 22. Cervical plate 22 may be positioned on a bone. Bone fastener 32 may be advanced into the bone until a surface of head 34 of bone fastener 32 contacts deflectable portions 101. The tapering of the outer surface of head 34 of bone fastener 32 provides a ramping force on deflectable portions 101 to deflect deflectable portions 101 radially outwards as bone fastener 32 is advanced into the bone. In some embodiments, outer surface 46 of ring 30 may contact surface 48 of interior space 24. Lower surface 28 of cervical plate 22 may contact a vertebra.

Referring to FIG. 12C, bone fastener 32 may be advanced through cervical plate 22 to the desired depth in the vertebra. Head 34 of bone fastener 32 may penetrate ring 30 such that head 34 passes through deflectable portions 101 or otherwise allows one or more deflectable portions 101 to return to a substantially non-deflected state. In some embodiments, one or more deflectable portions 101 may return to an un-deflected state. Driver head 456 of an insertion/extraction tool may be withdrawn from head 34 of bone fastener 32. After insertion, if bone fastener 32 becomes loose within the bone, backout of bone fastener 32 from cervical plate 22 may be resisted by flange 31 in contact with head 34 and flange 31 positioned in interior space 24. Thus, even if shank 54 loosens within the bone, head 34 will tend to remain within ring 30 in interior space 24 of cervical plate 22 so as not to protrude from cervical plate 22 into surrounding body tissue. In some embodiments, there may be some freedom of movement in the connection between flange 31 and interior space 24 to allow bone fastener 32 to back out slightly from a bone after insertion. In some embodiments, the freedom of movement is limited so that head 34 may not protrude from cervical plate 22.

After bone fastener 32 has been secured to the vertebra and, driver head 456 has been removed from tool portion 126, the polyaxial nature of ring 30 in interior space 24 may allow some rotation of bone fastener 32 relative to cervical plate 22. Ring 30 may also inhibit bone fastener 32 from backing out of the vertebral body.

Any of the embodiments described above may be used individually or in combination with other embodiments described above. Further modifications and alternative embodiments of various aspects of the disclosure will be apparent to those skilled in the art in view of this description. Accordingly, this description is to be construed as illustrative only and is for the purpose of teaching those skilled in the art the general manner of carrying out the disclosure. It is to be understood that the forms of the disclosure shown and described herein are to be taken as examples of embodiments. Elements and materials may be substituted for those illustrated and described herein, parts and processes may be reversed, and certain features of the disclosure may be utilized independently, all as would be apparent to one skilled in the art after having the benefit of this description of the disclosure. Changes may be made in the elements described herein without departing from the spirit and scope of the disclosure as set forth in the following claims.

What is claimed is:

1. A cervical plate comprising:
a plurality of boreholes through which the cervical plate is attachable to a vertebra via a plurality of bone fasteners; and
at least two openings through which a cortical region of the vertebra is visible when attaching the cervical plate to the vertebra;
wherein a first and a second of the plurality of boreholes are transversely aligned at a first end of the cervical plate;
wherein a first of the at least two openings has a first tip that reaches between the first and the second of the plurality of boreholes at first point on a midline of the cervical plate towards the first end of the cervical plate;
wherein a third and a fourth of the plurality of boreholes are transversely aligned at a second end of the cervical plate;
wherein a second of the at least two openings has a second tip that reaches between the third and the fourth of the plurality of boreholes at a second point on the midline of the cervical plate towards the second end of the cervical plate;
wherein each of the first, the second, the third, and the fourth of the plurality of boreholes has an interior space that accommodates a ring with a deflectable portion through which one of the plurality of bone fasteners is insertable.

2. The cervical plate of claim 1, wherein the ring is rotatable relative to the cervical plate within the interior space of the borehole.

3. The cervical plate of claim 1, wherein the interior space of the borehole allows the ring to be rotated to advance a bone fastener at a selected angle relative to the cervical plate, and wherein the bone fastener may be advanced through the ring to deflect the deflectable portion of the ring outward and the bone fastener may be further advanced through the opening to allow the deflectable portion of the ring to deflect inward to inhibit withdrawal of the bone fastener from the cervical plate.

4. The cervical plate of claim 1, wherein the deflectable portion comprises a plurality of tabs.

5. The cervical plate of claim 1, wherein at least one of the at least two openings has a pentagonal shape comprising:
a base side for alignment with an inferior or superior surface of a vertebra, wherein alignment of the base side with an inferior or superior surface of a vertebra positions the transversely aligned boreholes within the cortical region of the vertebra; and
a plurality of vertices, wherein alignment of the tip vertex relative to the midline of the vertebra aligns the cervical plate with the midline of the vertebra.

6. The cervical plate of claim 1, wherein at least one of the at least two openings has a rhombus shape, comprising:
a major axis extending between a first vertex and a second vertex, wherein alignment of the major axis with the midline of the vertebra aligns the cervical plate with the midline of the vertebra; and
a minor axis extending between a third vertex and a fourth vertex, wherein alignment of the minor axis with an inferior or superior surface of a vertebra positions the transversely aligned boreholes within the cortical region of the vertebra.

7. The cervical plate of claim 1, wherein at least one of the at least two openings has a triangular shape comprising:
a base side, wherein alignment of the base side with an inferior or superior surface of a vertebra positions the transversely aligned boreholes within the cortical region of the vertebra; and
a tip vertex, wherein alignment of the tip vertex relative to the midline aligns the cervical plate with the midline of the vertebra.

8. A cervical plate comprising:
a plurality of boreholes through which the cervical plate is attachable to a vertebra via a plurality of bone fasteners; and
at least one alignment opening through which a cortical region of the vertebra is visible when attaching the cervical plate to the vertebra;
wherein a first and a second of the plurality of boreholes are transversely aligned at a first end of the cervical plate;
wherein the at least one alignment opening has a first tip that reaches between the first and the second of the plurality of boreholes at a first point on a midline of the cervical plate towards the first end of the cervical plate;
wherein a third and a fourth of the plurality of boreholes are transversely aligned at a second end of the cervical plate;
wherein the at least one alignment opening has a second tip that reaches between the third and the fourth of the plurality of boreholes at a second point on the midline of the cervical plate towards the second end of the cervical plate; and
wherein each of the first, the second, the third, and the fourth of the plurality of boreholes has an interior space that accommodates a ring with a deflectable portion through which one of the plurality of bone fasteners is insertable.

9. The cervical plate of claim 8, wherein the ring is rotatable relative to the cervical plate within the interior space of the borehole.

10. The cervical plate of claim 9, wherein the interior space of the borehole allows the ring to be rotated to advance the bone fastener at a selected angle relative to the cervical plate, and wherein the bone fastener may be advanced through the ring to deflect the deflectable portion of the ring outward and the bone fastener may be further advanced through the opening to allow the deflectable portion of the ring to deflect inward to inhibit withdrawal of the bone fastener from the cervical plate.

11. The cervical plate of claim 8, wherein the deflectable portion comprises a plurality of tabs.

12. The cervical plate of claim 8, wherein at least one of the at least one openings has a pentagonal shape comprising:
a base side for alignment with an inferior or superior surface of a vertebra, wherein alignment of the base side with an inferior or superior surface of a vertebra positions the transversely aligned boreholes within the cortical region of the vertebra; and
a plurality of vertices, wherein alignment of the tip vertex relative to the midline of the vertebra aligns the cervical plate with the midline of the vertebra.

13. The cervical plate of claim 8, wherein at least one of the at least one openings has a rhombus shape, comprising:
a major axis extending between a first vertex and a second vertex, wherein alignment of the major axis with the midline of the vertebra aligns the cervical plate with the midline of the vertebra; and
a minor axis extending between a third vertex and a fourth vertex, wherein alignment of the minor axis with an inferior or superior surface of a vertebra positions the transversely aligned boreholes within the cortical region of the vertebra.

14. The cervical plate of claim 8, wherein an opening has a triangular shape comprising:
a base side, wherein alignment of the base side with an inferior or superior surface of a vertebra positions the transversely aligned boreholes within the cortical region of the vertebra; and
a tip vertex, wherein alignment of the tip vertex relative to the midline aligns the cervical plate with the midline of the vertebra.

15. A method for stabilizing a cervical portion of a spine, comprising:
making an incision in a patient;
advancing a cervical plate into the patient via the incision, wherein the cervical plate comprises:
a plurality of boreholes through which the cervical plate is attachable to a vertebra via a plurality of bone fasteners; and
at least one opening through which a cortical region of the vertebra is visible when attaching the cervical plate to the vertebra;
wherein a first and a second of the plurality of boreholes are transversely aligned at a first end of the cervical plate;
wherein the at least one opening has a first tip that reaches between the first and the second of the plurality of boreholes at a first point on a midline of the cervical plate towards the first end of the cervical plate; and
wherein each of the first and the second of the plurality of boreholes has an interior space that accommodates a ring with a deflectable portion through which one of the plurality of bone fasteners is insertable;
positioning the first end opening relative to a feature of a first vertebra;
advancing a plurality of bone fasteners via the transversely aligned boreholes into the cortical section of the first vertebra;
positioning the second end opening relative to a feature of a second vertebra; and
advancing a plurality of bone fasteners via the transversely aligned boreholes into the cortical section of the second vertebra.

16. The method of claim 15, wherein the method comprises minimally-invasive surgery.

17. The method of claim 15, wherein advancing a bone fastener via a transversely aligned borehole comprises rotating the ring in the borehole for advancement of the bone fastener at a selected angle relative to the cervical plate.

18. The method of claim 15, wherein at least one of the first end opening or second end opening has a pentagonal shape, wherein the method comprises:
aligning a first feature of the vertebra with a first side of the first end opening or a second end opening, wherein the transversely aligned boreholes are positioned within the cortical region of the vertebra; and
aligning a second feature of the vertebra with the tip vertex of the first end opening or the second end opening, wherein the midline of the cervical plate is aligned or substantially aligned with the midline of the vertebra.

19. The method of claim 15, wherein at least one of the first end opening or the second end opening has a triangular shape, wherein the method comprises:
aligning a first feature of the vertebra with a first side of the first end opening or the second end opening, wherein the transversely aligned boreholes are positioned within the cortical region of the vertebra; and
aligning a second feature of the vertebra with the tip vertex of the first end opening or the second end opening, wherein the midline of the cervical plate is aligned or substantially aligned with the midline of the vertebra.

20. The method of claim 15, wherein at least one of the first end opening or second end opening has a rhombus shape, wherein the method comprises:
aligning a first feature of the vertebra with the major axis of the rhombus, wherein the midline of the cervical plate is aligned or substantially aligned with the midline of the vertebra; and
aligning a second feature of the vertebra with a second tip of the first end opening or the second end opening, wherein the transversely aligned boreholes are positioned within the cortical region of the vertebra.
